(19) United States
(12) Patent Application Publication
(10) Pub. No.: US 2010/0286477 A1
(43) Pub. Date: Nov. 11, 2010

(54) INTERNAL TISSUE VISUALIZATION SYSTEM COMPRISING A RF-SHIELDED VISUALIZATION SENSOR MODULE

(76) Inventors: Xiaolong OuYang, Palo Alto, CA (US); James S. Cybulski, Menlo Park, CA (US); Eric Shultz, Menlo Park, CA (US); Fred R. Seddiqui, Los Altos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/437,865

(22) Filed: May 8, 2009

(51) Int. Cl. A61B 1/04

(52) U.S. Cl. 600/109

(57) ABSTRACT

Internal tissue visualization devices having RF-shielded visualization sensor modules are provided. Also provided are systems that include the devices, as well as methods of visualizing internal tissue of a subject using the tissue visualization devices and systems.

Traditional surgical procedures, both therapeutic and diagnostic, for pathologies located within the body can cause significant trauma to the intervening tissues. These procedures often require a long incision, extensive muscle stripping, prolonged retraction of tissues, denervation and devascularization of tissue. These procedures can require operating room time of several hours and several weeks of post-operative recovery time due to the destruction of tissue during the surgical procedure. In some cases, these invasive procedures lead to permanent scarring and pain that can be more severe than the pain leading to the surgical intervention.

The development of percutaneous procedures has yielded a major improvement in reducing recovery time and post-operative pain because minimal dissection of tissue, such as muscle tissue, is required. For example, minimally invasive surgical techniques are desirable for spinal and neurosurgical applications because of the need for access to locations within the body and the danger of damage to vital intervening tissues. While developments in minimally invasive surgery are steps in the right direction, there remains a need for further development in minimally invasive surgical instruments and methods.

### SUMMARY

Internal tissue visualization devices having RF-shielded visualization sensor modules are provided. Also provided are systems that include the devices, as well as methods of visualizing internal tissue of a subject using the tissue visualization devices and systems.

### BRIEF DESCRIPTION OF THE FIGURES

FIG. 1 shows a CMOS visualization sub-system that may be incorporated into a tissue modification system according to an embodiment of the invention.

FIGS. 2A and 2B provide two different views of a disposable tissue visualization and modification device according to an embodiment of the invention.

FIG. 3 provides a view of the distal end of a device according to one embodiment of the invention.

### DETAILED DESCRIPTION

Internal tissue visualization devices having RF-shielded visualization sensor modules are provided. Also provided are systems that include the devices, as well as methods of visualizing internal tissue of a subject using the tissue visualization devices and systems.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, aspects of embodiments of the subject tissue visualization devices and systems are described first in greater detail. Next, embodiments of methods of visualizing an internal target tissue of a subject in which the subject tissue visualization systems may find use are reviewed in greater detail.

### Tissue Visualization Devices and Systems

As summarized above, aspects of the invention include internal tissue visualization systems. The internal tissue visualization systems are visualization systems that are configured to visualize an internal tissue site of a subject. As such, the systems are structured or designed to provide images of a tissue site inside of a body, such as a living body, to a user. As such, aspects of systems of the invention include internal tissue visualization devices that are useful for visualizing an internal target tissue site, e.g., a spinal location that is near or inside of an intervertebral disc (IVD). The internal tissue visualization devices of embodiments of systems of the invention are dimensioned such that at least the distal end of the devices can pass through a minimally invasive body opening. As such, at least the distal end of the devices of these embodiments may be introduced to an internal target site of a patient, e.g., a spinal location that is near or inside of an intervertebral disc, through a minimal incision, e.g., one that is less than the size of an incision employed for an access device having a outer diameter of 20 mm or smaller, e.g., less than 75% the size of such an incision, such as less than 50% of the size of such an incision, or smaller. In some instances, at least the distal end of the elongated member of the devices is dimensioned to pass through a Cambin's triangle. The Cambin's triangle (also known in the art as the Pambin's triangle) is an anatomical spinal structure bounded by an exiting nerve root and a traversing nerve root and a disc. The exiting root is the root that leaves the spinal canal just cephalad (above) the disc, and the traversing root is the root that leaves the spinal canal just caudad (below) the disc. Where the distal end of the elongated member is dimensioned to pass through a Cambin's triangle, at least the distal end of the device has a longest cross-sectional dimension that is 10 mm or less, such as 8 mm or less and including 7 mm or less. In some instances, the elongated member has an outer diameter that is 7.5 mm or less, such as 7.0 mm or less, including 6.7 mm or less, such as 6.6 mm or less, 6.5 mm or less, 6.0 mm or less, 5.5 mm or less, 5.0 mm or less.

As summarized above, internal tissue visualization devices of the systems of the invention include an elongated member. As this component of the devices is elongated, it has a length that is 1.5 times or longer than its width, such as 2 times or longer than its width, including 5 or even 10 times or longer than its width, e.g., 20 times longer than its width, 30 times longer than its width, or longer. The length of the elongated member may vary, and in some instances ranges from 5 cm to 20 cm, such as 7.5 cm to 15 cm and including 10 to 12 cm. The elongated member may have the same outer cross-sectional dimensions (e.g., diameter) along its entire length. Alternatively, the cross-sectional diameter may vary along the length of the elongated member.

The elongated members of the subject tissue visualization devices have a proximal end and a distal end. The term “proximal end”, as used herein, refers to the end of the elongated member that is nearer the user (such as a physician operating the device in a tissue modification procedure), and the term “distal end”, as used herein, refers to the end of the elongated member that is nearer the internal target tissue of the subject during use. The elongated member is, in some instances, a structure of sufficient rigidity to allow the distal end to be pushed through tissue when sufficient force is applied to the proximal end of the elongate member. As such, in these embodiments the elongated member is not pliant or flexible, at least not to any significant extent.

As summarized above, the visualization devices include a RF-shielded visualization sensor module. The RF-shielded visualization sensor module is integrated with the elongated member. As the RF-shielded visualization sensor module is integrated with the elongated member, it cannot be removed from the remainder of the elongated member and device without significantly compromising the structure and functionality of the device. Accordingly, the devices of the present invention are distinguished from devices which include a “working channel” through which a separate autonomous device is passed through. In contrast to such devices, since the RF-shielded visualization sensor module of the present device is integrated with the elongated member, it is not a separate device from the elongated member that is merely present in a working channel of the elongated member and which can be removed from the working channel of such an elongated member without structurally compromising the elongated member in any way. The visualization sensor module may be integrated with the elongated member by a variety of different configurations. Integrated configurations include configurations where the visualization sensor of the visualization sensor module is fixed relative to the distal end of the elongated member, as well as configurations where the visualization sensor of the visualization sensor module is movable to some extent relative to the distal end of the elongated member. Movement of the visualization sensor of the visualization sensor module may also be provided relative to the distal end of the elongated member, but then fixed with respect to another component present at the distal end, such as a distal end integrated tissue modifier. Specific configurations of interest are further described below in connection with the figures.

As summarized above, devices of the invention include integrated RF-shielded visualization sensor modules. As the visualization sensor module is RF-shielded, the visualization sensor module includes an RF shield that substantially inhibits, if not completely prevents, an ambient RF field from reaching and interacting with circuitry of the visualization sensor. As such, the RF shield is a structure which substantially inhibits, if not completely prevents, ambient RF energy (e.g., as provided by a distal end RF electrode, as described in greater detail blow) from impacting the circuitry function of the visualization sensor.

Visualization sensor modules of devices of the invention include at least a visualization sensor. In certain embodiments, the devices may further include a conductive member that conductively connects the visualization sensor with another location of the device, such as a proximal end location. Additional components may also be present in the visualization sensor module, where these components are described in greater detail below.

The RF shield of the visualization sensor module may have a variety of different configurations. The RF shield may include an enclosure element or elements which serve to shield the circuitry of the visualization sensor from an ambient RF field. In some instances, the RF shield is a grounded conductive enclosure component or components which are associated with the visualization sensor, conductive member and other components of the visualization sensor module. In some instances, the visualization sensor of the visualization sensor module is present in a housing, where the housing may include a grounder outer conductive layer which serves as an RF shield component. In these instances, the RF shield is an outer grounded conductive layer. The conductive enclosure or enclosures of the RF-shielded visualization sensor module may be fabricated from a variety of different conductive materials, such as metals, metal alloys, etc., where specific conductive materials of interest include, but are not limited to: copper foils and the like. In certain instances, the RF shield is a metallic layer. This layer, when present, may vary in thickness, but in some instances has a thickness ranging from 0.2 mm to 0.7 mm, such as 0.3 mm to 0.6 mm and including 0.4 mm to 0.5 mm.

As reviewed above, visualization sensor modules of the invention include visualization sensors. Visualization sensors of interest include miniature imaging sensors that have a cross-sectional area which is sufficiently small for its intended use and yet retains a sufficiently high matrix resolution. Imaging sensors of interest are those that include a photosensitive component, e.g., array of photosensitive elements that convert light into electrons, coupled to a circuitry component, such as an integrated circuit. The integrated circuit may be configured to obtain and integrate the signals from the photosensitive array and output image data, which image data may in turn be conveyed to an extra-corporeal device configured to receive the data and display it to a user. The image sensors of these embodiments may be viewed as integrated circuit image sensors. The integrated circuit component of these sensors may include a variety of different types of functionalities, including but not limited to: image signal processing, memory, and data transmission circuitry to transmit data from the visualization sensor to an extra-corporeal location, etc. The miniature imaging sensors may further include a lens component made up of one or more lenses positioned relative to the photosensitive component so as to focus images on the photosensitive component. Specific types of miniature imaging sensors of interest include complementary metal-oxide-semiconductor (CMOS) sensors and charge-coupled device (CCD) sensors. The sensors may have any convenient configuration, including circular, square, rectangular, etc. Visualization sensors of interest may have a longest cross-sectional dimension that varies depending on the particular embodiment, where in some instances the longest cross sectional dimension (e.g., diameter) is 4.0 mm or less, such as 3.5 mm or less, including 3.0 mm or less, such as 2.5 mm or less, including 2.0 mm or less, including 1.5 mm or less, including 1.0 mm or less.

Imaging sensors of interest may be either frontside or backside illumination sensors, and have sufficiently small dimensions while maintaining sufficient functionality to be integrated at the distal end of the elongated members of the devices of the invention. Aspects of these sensors are further described in one or more the following U.S. patents, the disclosures of which are herein incorporated by reference: U.S. Pat. Nos. 7,388,242; 7,368,772; 7,355,228; 7,345,330; 7,344,910; 7,268,335; 7,209,601; 7,196,314; 7,193,198; 7,161,130; and 7,154,137.

In some instances, the visualization sensor is located at the distal end of the elongated member, such that the visualization sensor is a distal end visualization sensor. In these instances, the visualization sensor is located at or near the distal end of the elongated member. Accordingly, it is positioned at 3 mm or closer to the distal end, such as at 2 mm or closer to the distal end, including at 1 mm or closer to the distal end. In some instances, the visualization sensor is located at the distal end of the elongated member. The visualization sensor may provide for front viewing and/or side-viewing, as desired. Accordingly, the visualization sensor may be configured to provide image data as seen in the forward direction from the distal end of the elongated member. Alternatively, the visualization sensor may be configured to provide image data as seen from the side of the elongate member. In yet other embodiments, a visualization sensor may be configured to provide image data from both the front and the side, e.g., where the image sensor faces at an angle that is less than 90° relative to the longitudinal axis of the elongated member.

Components of the visualization sensor, e.g., the integrated circuit, one or more lenses, etc., may be present in a housing. The housing may have any convenient configuration, where the particular configuration may be chosen based on location of the sensor, direction of view of the sensor, etc. The housing may be fabricated from any convenient material. In some instances, non-conductive materials, e.g., polymeric materials, are employed.

Visualization sensor modules of devices of the invention may further include functionality for conveying image data to an extra-corporeal device, such as an image display device, of a system. In some instances, a signal cable (or other type of signal conveyance element) may be present to connect the image sensor at the distal end to a device at the proximal end of the elongate member, e.g., in the form of one or more wires running along the length of the elongate member from the distal to the proximal end. In some instances, the visualization sensor is coupled to a conductive member (e.g., cable or analogous structure) that conductively connects the visualization sensor to a proximal end location of the elongated member, where each of these components are present in a conductive enclosure which serves as a RF shield for these components of the visualization sensor module. Alternatively, wireless communication protocols may be employed, e.g., where the imaging sensor is operatively coupled to a wireless data transmitter, which may be positioned at the distal end of the elongated member (including integrated into the visualization sensor, at some position along the elongated member or at the proximal end of the device, e.g., at a location of the proximal end of the elongated member or associated with the handle of the device).

Where desired, the devices may include one or more illumination elements configured to illuminate a target tissue location so that the location can be visualized with a visualization sensor, e.g., as described above. A variety of different types of light sources may be employed as illumination elements (also referred to herein as illuminators), so long as their dimensions are such that they can be positioned at the distal end of the elongated member. The light sources may be integrated with a given component (e.g., elongated member) such that they are configured relative to the component such that the light source element cannot be removed from the remainder of the component without significantly compromising the structure of the component. As such, the integrated illumination element of these embodiments is not readily removable from the remainder of the component, such that the illumination element and remainder of the component form an inter-related whole. The light sources may be light emitting diodes configured to emit light of the desired wavelength range, or optical conveyance elements, e.g., optical fibers, configured to convey light of the desired wavelength range from a location other than the distal end of the elongate member, e.g., a location at the proximal end of the elongate member, to the distal end of the elongate member.

As with the image sensors, the light sources may include a conductive element, e.g., wire, or an optical fiber, which runs the length of the elongate member to provide for power and control of the light sources from a location outside the body, e.g., an extracorporeal control device. In some embodiments, the devices are configured such that the RF shielded visualization sensor and the light emitting diode are integrated with the RF-shielded visualization sensor, such that they are coupled to a common RF shielded conductive member that conductively connects the visualization sensor to a proximal end location of the elongated member.

Where desired, the light sources may include a diffusion element to provide for uniform illumination of the target tissue site. Any convenient diffusion element may be employed, including but not limited to a translucent cover or layer (fabricated from any convenient translucent material) through which light from the light source passes and is thus diffused. In those embodiments of the invention where the system includes two or more illumination elements, the illumination elements may emit light of the same wavelength or they may be spectrally distinct light sources, where by “spectrally distinct” is meant that the light sources emit light at wavelengths that do not substantially overlap, such as white light and infra-red light. In certain embodiments, an illumination configuration as described in copending U.S. application Ser. Nos. 12/269,770 and 12/269,772 (the disclosures of which are herein incorporated by reference) is present in the device.

Depending on the particular device embodiment, the elongated member may or may not include one or more lumens that extend at least partially along its length. When present, the lumens may vary in diameter and may be employed for a variety of different purposes, such as irrigation, aspiration, electrical isolation (for example of conductive members, such as wires), as a mechanical guide, etc., as reviewed in greater detail below. When present, such lumens may have a longest cross section that varies, ranging in some instances from 0.5 to 5.0 mm, such as 1.0 to 4.5 mm, including 1.0 to 4.0 mm. The lumens may have any convenient cross-sectional shape, including but not limited to circular, square, rectangular, triangular, semi-circular, trapezoidal, irregular, etc., as desired. These lumens may be provided for a variety of different functions, including as irrigation and/or aspiration lumens, as described in greater detail below.

Where desired, devices of the invention may further include a distal end tissue modifier. Tissue modifiers are components that interact with tissue in some manner to modify the tissue in a desired way. The term modify is used broadly to refer to changing in some way, including cutting the tissue, ablating the tissue, delivering an agent(s) to the tissue, freezing the tissue, etc. As such, of interest as tissue modifiers are tissue cutters, tissue ablators, tissue freezing/heating elements, agent delivery devices, etc. Tissue cutters of interest include, but are not limited to: blades, liquid jet devices, lasers and the like. Tissue ablators of interest include, but are not limited to ablation devices, such as devices for delivery ultrasonic energy (e.g., as employed in ultrasonic ablation), devices for delivering plasma energy, devices for delivering radiofrequency (RF) energy, devices for delivering microwave energy, etc. Energy transfer devices of interest include, but are not limited to: devices for modulating the temperature of tissue, e.g., freezing or heating devices, etc.

In some instances, the tissue modifier includes at least one electrode. For example, tissue modifiers of interest may include RF energy tissue modifiers, which include at least one electrode and may be configured in a variety of different ways depending on the desired configuration of the RF circuit. An RF circuit can be completed substantially entirely at target tissue location of interest (bipolar device) or by use of a second electrode attached to another portion of the patient's body (monopolar device). In either case, a controllable delivery of RF energy is achieved. Aspects of the subject tissue modification devices include a radiofrequency (RF) electrode positioned at the distal end of the elongated member. RF electrodes are devices for the delivery of radiofrequency energy, such as ultrasound, microwaves, and the like. In some instances, the RF electrode is an electrical conductor for delivering RF energy to a particular location, such as a desired target tissue. For instance, in certain cases, the RF electrode can be an RF ablation electrode. RF electrodes of the subject tissue modification devices can include a conductor, such as a metal wire, and can be dimensioned to access an intervertebral disc space. RF electrodes may be shaped in a variety of different formats, such as circular, square, rectangular, oval, etc. The dimensions of such electrodes may vary, where in some embodiments they RF electrode has a longest cross-sectional dimension that is 7 mm or less, 6 mm or less 5 mm or less, 4 mm or less, 3 mm or less or event 2 mm or less, as desired. Where the electrode includes a wire, the diameter of the wire in such embodiments may be 180 μm, such as 150 μm or less, such as 130 μm or less, such as 100 μm or less, such as 80 μm or less. A variety of different RF electrode configurations suitable for use in tissue modification and include, but are not limited to, those described in U.S. Pat. Nos. 7,449,019; 7,137,981; 6,997,941; 6,837,887; 6,241,727; 6,112,123; 6,607,529; 5,334,183. RF electrode systems or components thereof may be adapted for use in devices of the present invention (when coupled with guidance provided by the present specification) and, as such, the disclosures of the RF electrode configurations in these patents are herein incorporated by reference. Specific RF electrode configurations of interest are further described in connection with the figures, below, as well as in U.S. Provisional application Ser. No. 12/422,176; the disclosure of which is herein incorporated by reference.

In some instances, the tissue modifier is integrated at the distal end of the elongated member. In these embodiments, as the tissue modifier is integrated at the distal end of the device, it cannot be entirely removed from the remainder of the device without significantly compromising the structure and functionality of the device. While the tissue modifier cannot entirely be removed from the device without compromising the structure and functionality of the device, components of the tissue modifier may be removable and replaceable. For example, a RF electrode tissue modifier may be configured such that the wire component of the tissue modifier may be replaceable while the remainder of the tissue modifier is not. Accordingly, the devices of the present invention are distinguished from devices which include a “working channel” through which a separate autonomous tissue modifier device, such as an autonomous RF electrode device, is passed through. In contrast to such devices, since the tissue modifier of the present device is integrated at the distal end, it is not a separate device from the elongated member that is merely present in a working channel of the elongated member and which can be removed from the working channel of such an elongated member without structurally compromising the elongated member in any way. The tissue modifier may be integrated with the distal end of the elongated member by a variety of different configurations. Integrated configurations include configurations where the tissue modifier is fixed relative to the distal end of the elongated member, as well as configurations where the tissue modifier is movable to some extent relative to the distal end of the elongated member may be employed in devices of the invention. Specific configurations of interest are further described below in connection with the figures. As the tissue modifier is a distal end integrated tissue modifier, it is located at or near the distal end of the elongated member. Accordingly, it is positioned at 10 mm or closer to the distal end, such as at 5 mm or closer to the distal end, including at 2 mm or closer to the distal end. In some instances, the tissue modifier is located at the distal end of the elongated member.

Depending on the nature of the tissue modifier, the devices will include proximal end connectors for operatively connecting the device and tissue modifier to extra-corporeal elements required for operability of the tissue modifier, such as extra-corporeal RF controllers (e.g., RF tuners), mechanical tissue cutter controllers, liquid jet controllers, etc.

In some embodiments, an integrated articulation mechanism that imparts steerability to at least one of the visualization sensor, the tissue modifier and the distal end of the elongated member is also present in the device. By “steerability” is meant the ability to maneuver or orient the visualization sensor, tissue modifier and/or distal end of the elongated member as desired during a procedure, e.g., by using controls positioned at the proximal end of the device. In these embodiments, the devices include a steerability mechanism (or one or more elements located at the distal end of the elongated member) which renders the desired distal end component maneuverable as desired through proximal end control. As such, the term “steerability”, as used herein, refers to a mechanism that provides a user steering functionality, such as the ability to change direction in a desired manner, such as by moving left, right, up or down relative to the initial direction. The steering functionality can be provided by a variety of different mechanisms. Examples of suitable mechanisms include, but are not limited to one or more wires, tubes, plates, meshes or combinations thereof, made from appropriate materials, such as shape memory materials, music wire, etc. In some instances, the distal end of the elongated member is provided with a distinct, additional capability that allows it to be independently rotated about its longitudinal axis when a significant portion of the operating handle is maintained in a fixed position, as discussed in greater detail below. The extent of distal component articulations of the invention may vary, such as from −180 to +180°; e.g., −90 to +90°. Alternatively, the distal probe tip articulations may range from 0 to 360°, such as 0 to +180°, and including 0 to +90°, with provisions for rotating the entire probe about its axis so that the full range of angles is accessible on either side of the axis of the probe, e.g., as described in greater detail below. Articulation mechanisms of interest are further described in published PCT Application Publication Nos. WO 2009029639; WO 2008/094444; WO 2008/094439 and WO 2008/094436; the disclosures of which are herein incorporated by reference. Specific articulation configurations of interest are further described in connection with the figures, below, as well as in U.S. application Ser. No. 12/422,176; the disclosure of which is herein incorporated by reference.

In certain embodiments, devices of the invention may further include an irrigator and aspirator configured to flush an internal target tissue site and/or a component of the device, such as a lens of the visualization sensor. As such, the elongated member may further include one or more lumens that run at least the substantial length of the device, e.g., for performing a variety of different functions, as summarized above. In certain embodiments where it is desired to flush (i.e., wash) the target tissue site at the distal end of the elongated member (e.g. to remove ablated tissue from the location, etc.), the elongated member may include both irrigation lumens and aspiration lumens. Thus, the tissue modification device can comprise an irrigation lumen located at the distal end of the elongated member, and the tissue modification device can include an aspiration lumen located at the distal end of the elongated member. During use, the irrigation lumen is operatively connected to a fluid source (e.g., a physiologically acceptable fluid, such as saline) at the proximal end of the device, where the fluid source is configured to introduce fluid into the lumen under positive pressure, e.g., at a pressure ranging from 0 psi to 60 psi, so that fluid is conveyed along the irrigation lumen and out the distal end. While the dimensions of the irrigation lumen may vary, in certain embodiments the longest cross-sectional dimension of the irrigation lumen ranges from 0.5 mm to 5 mm, such as 0.5 mm to 3 mm, including 0.5 mm to 1.5 mm. During use, the aspiration lumen is operatively connected to a source of negative pressure (e.g., a vacuum source) at the proximal end of the device. While the dimensions of the aspiration lumen may vary, in certain embodiments the longest cross-sectional dimension of the aspiration lumen ranges from 1 mm to 7 mm, such as 1 mm to 6 mm, including 1 mm to 5 mm. In some embodiments, the aspirator comprises a port having a cross-sectional area that is 33% or more, such as 50% or more, including 66% or more, of the cross-sectional area of the distal end of the elongated member. In some instances, the negative pressure source is configured to draw fluid and/or tissue from the target tissue site at the distal end into the aspiration lumen under negative pressure, e.g., at a negative pressure ranging from 300 to 600 mmHg, such as 550 mmHg, so that fluid and/or tissue is removed from the tissue site and conveyed along the aspiration lumen and out the proximal end, e.g., into a waste reservoir. In certain embodiments, the irrigation lumen and aspiration lumen may be separate lumens, while in other embodiments, the irrigation lumen and the aspiration lumen can be included in a single lumen, for example as concentric tubes with the inner tube providing for aspiration and the outer tube providing for irrigation. When present, the lumen or lumens of the flushing functionality of the device may be operatively coupled to extra-corporeal irrigation devices, such as a source of fluid, positive and negative pressure, etc. Where desired, irrigators and/or aspirators may be steerable, as described above. Examples of irrigators and aspirators of interest are provided below in greater detail in connection with certain of the figures, as well as in U.S. application Ser. No. 12/422,176; the disclosure of which is herein incorporated by reference.

Where desired, the devices may include a control structure, such as a handle, operably connected to the proximal end of the elongated member. By “operably connected” is meant that one structure is in communication (for example, mechanical, electrical, optical connection, or the like) with another structure. When present, the control structure (e.g., handle) is located at the proximal end of the device. The handle may have any convenient configuration, such as a hand-held wand with one or more control buttons, as a hand-held gun with a trigger, etc., where examples of suitable handle configurations are further provided below.

In some embodiments, the distal end of the elongated member is rotatable about its longitudinal axis when a significant portion of the operating handle is maintained in a fixed position. As such, at least the distal end of the elongated member can turn by some degree while the handle attached to the proximal end of the elongated member stays in a fixed position. The degree of rotation in a given device may vary, and may range from 0 to 360°, such as 0 to 270°, including 0 to 180°.

Devices of the invention may be disposable or reusable. As such, devices of the invention may be entirely reusable (e.g., be multi-use devices) or be entirely disposable (e.g., where all components of the device are single-use). In some instances, the device can be entirely reposable (e.g., where all components can be reused a limited number of times). Each of the components of the device may individually be single-use, of limited reusability, or indefinitely reusable, resulting in an overall device or system comprised of components having differing usability parameters.

Devices of the invention may be fabricated using any convenient materials or combination thereof including but not limited to: metallic materials such as tungsten, stainless steel alloys, platinum or its alloys, titanium or its alloys, molybdenum or its alloys, and nickel or its alloys, etc; polymeric materials, such as polytetrafluoroethylene, polyimide, PEEK, and the like; ceramics, such as alumina (e.g., STEATITE™ alumina, MAECOR™ alumina), etc.

Systems of the invention further include an extra-corporeal control unit operatively coupled to the proximal end of the elongated member. Extra-corporeal control units may include a number of different components, such as power sources, irrigation sources, aspiration sources, image data processing components, image display components (such as monitors, printers, and the like) for displaying to a user images obtained by the visualization sensor, data processors, e.g., in the form of computers, data storage devices, e.g., floppy disks, hard drives, CD-ROM, DVD, flash memory, etc., device and system controls, etc.

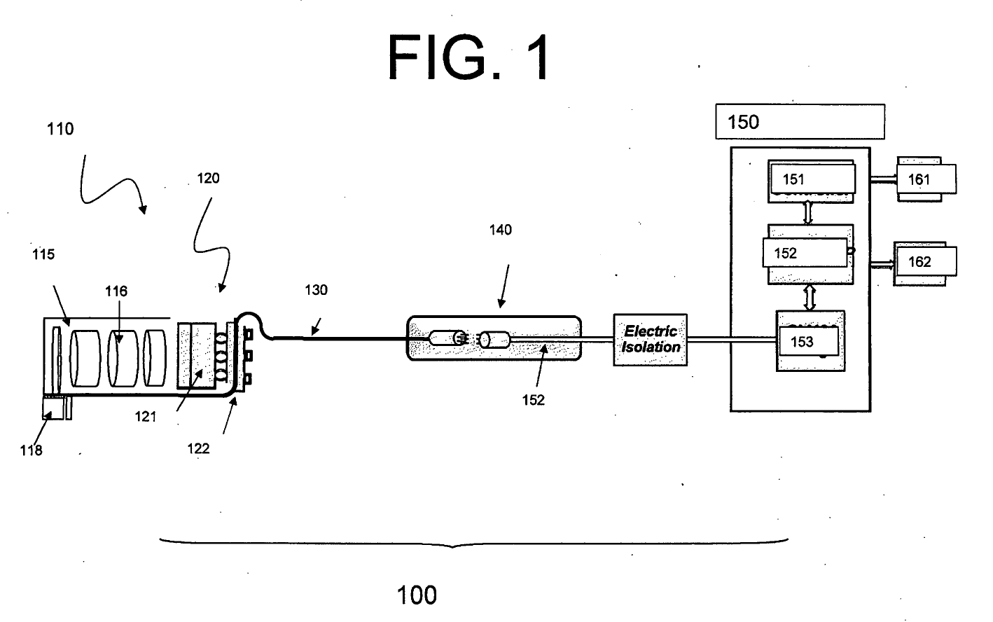

Within a given system, the RF-shielded visualization module may have a variety of different configurations. FIG. 1 provides an example of an embodiment of an integrated RF-shielded visualization module that includes a distal end CMOS visualization sensor and a flexible cable connecting the sensor to the proximal end of the device. As shown in FIG. 1, visualization sensor component 100 includes distal end CMOS visualization sensor 110 that includes lens housing 115 component operatively coupled to integrated circuit component 120. As shown in the figure, lens housing 115 includes a lens set 116. Also shown at the distal end is LED 118 which provides illumination for a target tissue location during use. Integrated circuit component 120 includes CMOS sensor integrated circuit 121 and rigid printed circuit board 122. The sub-components of lens housing/light source component 115 are operatively coupled to flexible cable 130 which provides for operative connection of the CMOS visualization sensor at the distal end of the device via the handle 140 to the video processing sub-system 150. In FIG. 1, the entire visualization sensor module (which includes the light source, visualization sensor and flexible cable) is shielded by a conductive outer layer on the visualization sensor housing and a metal tube that surrounds the flexible cable 130. These enclosures are connected and grounded to provide for RF-shielding of the circuitry components of the visualization sensor. They are also tied to the grounds of the RF circuitry which is associated with the RF electrode of the device (no shown). In the handle 140 the flexible cable operatively connects to a cable 152, which cable may have a grounded outer conductive layer that provides for RF isolation. RF shielded cable 152 connects to video processing sub-system 150 which includes a variety of functional blocks, such as host controller 151 (coupled to PC 161), digital signal processor 152 (coupled to LCD 162) and CMOS visualization sensor bridge 153. As shown in the system of FIG. 1 all the operative components of the visualization sensor, including the integrated circuit, as well as the LED, are operatively coupled to a common printed circuit board, which in turn is coupled to a signal RF shielded cable. This configuration provides numerous advantages in terms of device size, as well as cost and ease of manufacturing.

Systems of the invention may include a number of additional components in addition to the tissue modification devices and extra-corporeal control units, as described above. Additional components may include access port devices; root retractors; retractor devices, system component fixation devices; and the like; etc. Of interest are systems that further access devices as described in co-pending U.S. application Ser. Nos. 12/269,770; 12/269,772; and 12/269,775; the disclosures of which are herein incorporated by reference.

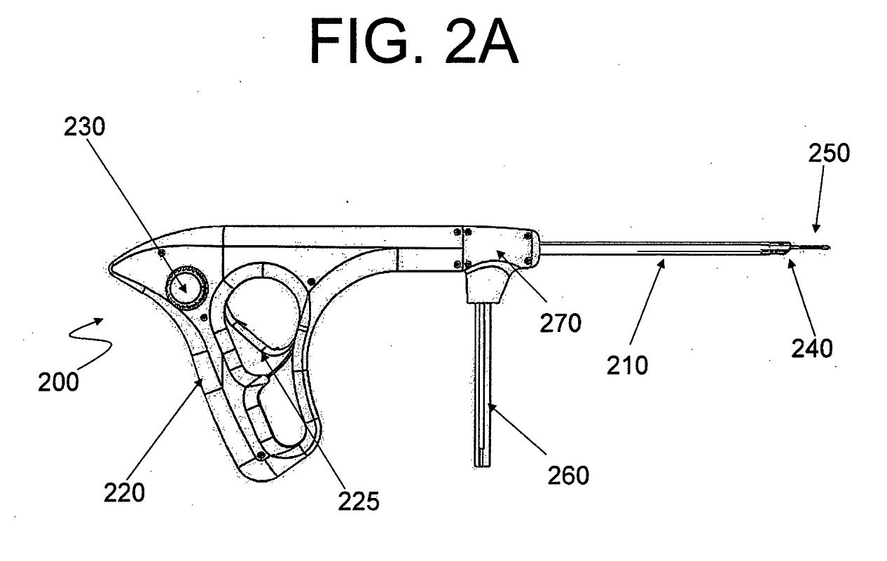

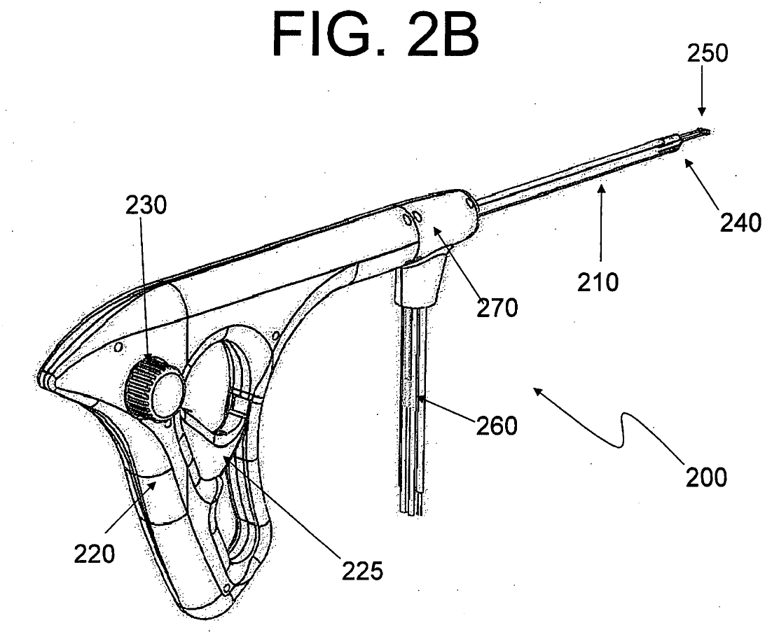

The systems of the invention may include a number of different types of visualization devices. An example of a visualization device is a handheld device as shown in FIGS. 2A and 2B, where the device shown in these figures includes, in addition to the RF shielded distal end integrated visualization sensor, a distal integrated RF electrode tissue modifier and irrigator and aspirator. FIGS. 2A and 2B provide two different side views of a device 200 according to one embodiment of the invention. Device 200 includes an elongated member 210 and an operating handle 220 at the proximal end of the elongated member 210. The operating handle has a gun configuration and includes a trigger 225 and thumbwheel 230 which provide a user with manual operation over certain functions of the device, e.g., RF electrode positioning and extension. Located at the distal end of the elongated member is an integrated RF-shielded visualization sensor 240 and tissue modifier 250. Control elements 260 (which may include aspiration and irrigation lumens, control/power wires, etc.) exit the handle 220 at the distal end region 270, which region 270 is rotatable relative to the remainder of the handle 220. A variety of additional components may be present at the distal end of the elongated member, which additional elements may include irrigators, aspirators, articulation mechanisms, etc. as described generally above.

With tissue modification devices of the invention that are configured to be hand-held, e.g., as shown in FIGS. 2A and 2B, the tissue modification devices may have a mass that is 1.5 kg or less, such as 1 kg or less, including 0.5 kg or less, e.g., 0.25 kg or less.

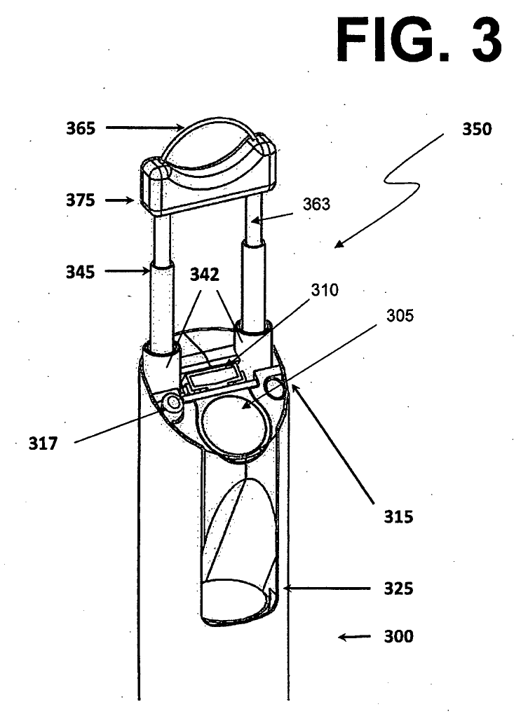

FIG. 3 provides a three-dimensional view of one embodiment of a distal end of tissue visualization device 300 (having a 6.5 mm outer dimension) of the invention. In FIG. 3, the distal end of the device includes an RF shielded integrated circular CMOS visualization sensor 305 and integrated LED 310. Also shown is a first forward facing irrigation lumen 315 and a second irrigation lumen 317 which is slightly extended from the distal end and is side facing so that fluid emitted from lumen 317 is flowed across CMOS visualization sensor 305 to clean the sensor of debris, when needed. Also shown is an aspiration lumen 325 positioned proximal the irrigation lumens 315 and 317 and integrated CMOS visualization sensor 305, where the aspiration lumen 325 is configured to aspirate fluid and tissue debris from a target tissue site during use. The distal end further includes an integrated steerable RF electrode assembly 350. RF electrode assembly 350 includes NITINOL shape memory guide tubes 345 extending from insulated (e.g., RF shielded) guide lumens 342. The RF electrode further includes a tungsten cutting wire 365 joined at each end to a NITINOL shape memory electrode wire 363 by a ceramic arc stop 375. As shown, the diameter of the cutting wire 365 is smaller than the diameter of the electrode wires 363, where the difference in size may vary and may range from 100 to 500 μm, such as 300 to 400 μm.

Additional embodiments of tissue modifiers and distal ends of tissue visualization devices of the invention may be found in U.S. application Ser. No. 12/422,176; the disclosure of which is herein incorporated by reference.

### Methods

Aspects of the subject invention also include methods of imaging (and in some embodiments modifying) an internal target tissue of a subject. Accordingly, aspects of the invention further include methods of imaging an internal tissue site with tissue visualization devices of the invention. A variety of internal tissue sites can be imaged with devices of the invention. In certain embodiments, the methods are methods of imaging an intervertebral disc in a minimally invasive manner. For ease of description, the methods are now primarily described further in terms of imaging IVD target tissue sites. However, the invention is not so limited, as the devices may be used to image a variety of distinct target tissue sites.

With respect to imaging an intervertebral disc or portion thereof e.g., exterior of the disc, nucleus pulposus, etc., embodiments of such methods include positioning a distal end of a minimally invasive intervertebral disc imaging device of the invention in viewing relationship to an intervertebral disc or portion of there, e.g., nucleus pulposus, internal site of nucleus pulposus, etc. By viewing relationship is meant that the distal end is positioned within 40 mm, such as within 10 mm, including within 5 mm of the target tissue site of interest. Positioning the distal end in viewing device in relation to the desired target tissue may be accomplished using any convenient approach, including through use of an access device, such as a cannula or retractor tube, which may or may not be fitted with a trocar, as desired. Following positioning of the distal end of the imaging device in viewing relationship to the target tissue, the target tissue, e.g., intervertebral disc or portion thereof, is imaged through use of the illumination and visualization elements to obtain image data. Image data obtained according to the methods of the invention is output to a user in the form of an image, e.g., using a monitor or other convenient medium as a display means. In certain embodiments, the image is a still image, while in other embodiments the image may be a video.

In certain embodiments, the methods include a step of tissue modification in addition to the tissue viewing. For example, the methods may include a step of tissue removal, e.g., using a combination of tissue cutting and irrigation or flushing. For example, the methods may include cuffing a least a portion of the tissue and then removing the cut tissue from the site, e.g., by flushing at least a portion of the imaged tissue location using a fluid introduced by an irrigation lumen and removed by an aspiration lumen.

The internal target tissue site may vary widely. Internal target tissue sites of interest include, but are not limited to, cardiac locations, vascular locations, orthopedic joints, central nervous system locations, etc. In certain cases, the internal target tissue site comprises spinal tissue.

The subject methods are suitable for use with a variety of mammals. Mammals of interest include, but are not limited to: race animals, e.g. horses, dogs, etc., work animals, e.g. horses, oxen etc., and humans. In some embodiments, the mammals on which the subject methods are practiced are humans.

### Utility

The subject tissue visualization devices and methods find use in a variety of different applications where it is desirable to image and/or modify an internal target tissue of a subject while minimizing damage to the surrounding tissue. The subject devices and methods find use in many applications, such as but not limited to surgical procedures, where a variety of different types of tissues may be removed, including but not limited to: soft tissue, cartilage, bone, ligament, etc. Specific procedures of interest include, but are not limited to, spinal fusion (such as Transforaminal Lumbar Interbody Fusion (TLIF)), total disc replacement (TDR), partial disc replacement (PDR), procedures in which all or part of the nucleus pulposus is removed from the intervertebral disc (IVD) space, arthroplasty, and the like. As such, methods of the invention also include treatment methods, e.g., where a disc is modified in some manner to treat an existing medical condition. Treatment methods of interest include, but are not limited to: annulotomy, nucleotomy, discectomy, annulus replacement, nucleus replacement, and decompression due to a bulging or extruded disc. Additional methods in which the imaging devices find use include those described in United States Published Application No. 20080255563.

In certain embodiments, the subject devices and methods facilitate the dissection of the nucleus pulposus while minimizing thermal damage to the surrounding tissue. In addition, the subject devices and methods can facilitate the surgeon's accessibility to the entire region interior to the outer shell, or annulus, of the IVD, while minimizing the risk of cutting or otherwise causing damage to the annulus or other adjacent structures (such as nerve roots) in the process of dissecting and removing the nucleus pulposus.

Furthermore, the subject devices and methods may find use in other procedures, such as but not limited to ablation procedures, including high-intensity focused ultrasound (HIFU) surgical ablation, cardiac tissue ablation, neoplastic tissue ablation (e.g. carcinoma tissue ablation, sarcoma tissue ablation, etc.), microwave ablation procedures, and the like. Yet additional applications of interest include, but are not limited to: orthopedic applications, e.g., fracture repair, bone remodeling, etc., sports medicine applications, e.g., ligament repair, cartilage removal, etc., neurosurgical applications, and the like.

### Kits

Also provided are kits for use in practicing the subject methods, where the kits may include one or more of the above devices, and/or components of the subject systems, as described above. The kit may further include other components, e.g., guidewires, access devices, fluid sources, etc., which may find use in practicing the subject methods. Various components may be packaged as desired, e.g., together or separately.

In addition to above mentioned components, the subject kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof are intended to encompass both structural and functional equivalents thereof. Additionally, it is intended that such equivalents include both currently known equivalents and equivalents developed in the future, i.e., any elements developed that perform the same function, regardless of structure. The scope of the present invention therefore, is not intended to be limited to the exemplary embodiments shown and described herein. Rather, the scope and spirit of present invention is embodied by the appended claims.

That which is claimed is:

1. An internal tissue visualization system, the system comprising:
(a) an internal tissue visualization device comprising:
(i) an elongated member having a proximal end and a distal end; and
(ii) an RF-shielded visualization sensor module; and
(b) an extra-corporeal control unit operatively coupled to the proximal end of the elongated member.

2. The system according to claim 1, wherein the RF-shielded visualization sensor module comprises a:
a visualization sensor comprising a lens and an integrated circuit, wherein the visualization sensor is integrated at the distal end of the elongated member; and
a grounded conductive enclosure that shields the integrated circuit from an RF field.

3. The tissue modification device according to claim 2, wherein the visualization sensor is a CMOS device.

4. The tissue modification device according to claim 2, wherein the visualization sensor is a CCD device.

5. The system according to claim 2, wherein the grounded conductive enclosure comprises a housing comprising an outer grounded conductive layer.

6. The system according to claim 5, wherein the outer grounded conductive layer is a metallic layer.

7. The system according to claim 2, wherein the RF-shielded visualization sensor module further comprises an RF-shielded conductive member that connects the visualization sensor to a proximal end location of the elongated member.

8. The system according to claim 1, wherein the distal end of the elongated member further comprises an integrated illuminator.

9. The system according to claim 8, wherein the illuminator is a light emitting diode.

10. The system according to claim 9, wherein the RF-shielded visualization sensor module comprises the light emitting diode.

11. The system according to claim 1, wherein the system further comprises a tissue modifier at the distal end of the elongated member.

12. The system according to claim 11, wherein the tissue modifier is integrated at the distal end.

13. The system according to claim 11, wherein the tissue modifier comprises an electrode.

14. The system according to claim 1, wherein the system comprises an image displayer for displaying to a user images obtained by the visualization sensor.

15. An internal tissue visualization device comprising:
an elongated member having a proximal end and a distal end; and
an RF-shielded visualization sensor module.

16. The device according to claim 15, wherein the RF-shielded visualization sensor module comprises:
a visualization sensor comprising a lens and an integrated circuit, wherein the visualization sensor is integrated at the distal end of the elongated member; and
a grounded conductive enclosure that shields the integrated circuit from an RF field.

17. The device according to claim 16, wherein the visualization sensor is a CMOS device.

18. The device according to claim 16, wherein the visualization sensor is a CCD device.

19. The device according to claim 16, wherein the grounded conductive enclosure comprises a housing comprising an outer grounded conductive layer.

20. The device according to claim 16, wherein the RF-shielded visualization sensor module further comprises an RF-shielded conductive member that connects the visualization sensor to a proximal end location of the elongated member.

21. The device according to claim 15, wherein the distal end of the elongated member further comprises an integrated illuminator.

22. The device according to claim 15, wherein the system further comprises a tissue modifier at the distal end of the elongated member.

23. The device according to claim 22, wherein the tissue modifier comprises an electrode.

24. A method of imaging an internal target tissue site of a subject, the method comprising:
(a) positioning the distal end of an internal tissue visualization device comprising:
(i) an elongated member having a proximal end and a distal end; and
(ii) an RF-shielded visualization sensor module;
in operable relation to the internal target tissue site; and
(b) visualizing the internal target tissue site with the RF-shielded visualization sensor module.

25. The method according to claim 24, wherein the internal target tissue site comprises spinal tissue.

26. The method according to claim 25, wherein the device further comprises a distal end tissue modifier and the method further comprises modifying tissue with the tissue modifier.
